(19) United States
(12) Patent Application Publication
(10) Pub. No.: US 2006/0271082 A1
(43) Pub. Date: Nov. 30, 2006

(54) Calibrated surgical probe

(76) Inventors: G. Lamar Kirchhevel, Laguna Niguel, CA (US); Richard L. Zaleski II, San Juan Capistrano, CA (US); Dean Y. Lin, Chino Hills, CA (US); Michael A. Zica, Costa Mesa, CA (US)

Correspondence Address: ALCON, IP LEGAL, TB4-8, 6201 SOUTH FREEWAY, FORT WORTH, TX, 76134, US

(21) Appl. No.: 11/442,469

(22) Filed: May 26, 2006

Related U.S. Application Data
(60) Provisional application No. 60/685,997, filed on May 31, 2005.

(51) Int. Cl. A61B 17/32

(52) U.S. Cl. 606/170

(57) ABSTRACT

A microsurgical probe tip and method of using same are disclosed. One embodiment of the microsurgical probe tip comprises: an outer cutting member, comprising a first tube having a wall, a closed end and a port formed in the wall near the closed end; an inner cutting member, comprising a second tube coaxial with and operable to move in a reciprocating motion within the first tube and having a first end operable to be coupled to a driving mechanism and a second end with a cutting edge for cutting tissue; a first alignment mark on the outer cutting member at a first predetermined position adjacent to the port; and a second alignment mark on the inner cutting member at a second predetermined position adjacent to the cutting edge of the inner cutting member, wherein the second alignment mark is visible through the port and operable to be aligned with the first alignment mark such that when the first and second alignment marks are aligned, a preferred relative positioning between the inner and outer cutting members is achieved. The microsurgical probe tip can further comprise one or more radial alignment marks on the inner cutting member, wherein the radial alignment marks are parallel to one another at fixed intervals from one another and positioned so that one or more of the radial alignment marks are visible through the port so as to indicate the relative lateral positioning between the inner cutting member and the outer cutting member. The radial alignment marks can be made by a method, or combination of methods, such as laser cutting, inkjet printing, and mechanical scribing. The driving mechanism can be a pneumatic driving mechanism, an electro-mechanical driving mechanism, and/or a magnetic driving mechanism. The microsurgical probe tip can further comprising a plurality of gauge marks on an outer surface of the outer cutting member, wherein the gauge marks are parallel to one another at a fixed interval (e.g., 1 millimeter) from one another and positioned so that the gauge marks can be used as a measuring tool in a surgical environment

### CROSS-REFERENCE TO RELATED APPLICATIONS

This application claims priority under 35 U.S.C. §119 to U.S. Provisional Patent Application No. 60/685,997, filed May 31, 2005, the entire contents of which are incorporated herein by reference.

### TECHNICAL FIELD OF THE INVENTION

The present invention relates generally to surgical instruments and, more particularly, to surgical instruments used in ophthalmic surgery. Even more particularly, the present invention relates to instruments, such as vitrectomy probes, suitable for use in performing micro-surgical procedures in the posterior portion of the eye.

### BACKGROUND OF THE INVENTION

Many micro-surgical procedures require precision cutting and/or removal of various body tissues. For example, certain ophthalmic procedures require the cutting and/or removal of the vitreous humor, a transparent jelly-like material that fills the posterior segment of the eye. The vitreous humor, or vitreous, is composed of numerous microscopic fibers that are often attached to the retina. Therefore, cutting and removal of the vitreous must be done with great care to avoid traction on the retina, the separation of the retina from the choroid, a retinal tear, or, in the worse case, cutting and removal of the retina itself.

The use of micro-surgical cutting probes in posterior segment ophthalmic surgery is well known. The instrument most commonly used and generally preferred for vitreous surgery is a pneumatically operated axial guillotine cutter (a “vitrectomy probe”). Such vitrectomy probes are typically inserted via an incision in the sclera near the pars plana. A surgeon might also insert other micro-surgical instruments, such as a fiber-optic illuminator, an infusion cannula, or an aspiration probe during the posterior segment surgery. The surgeon typically performs the procedure while viewing the eye under a microscope.

Conventional vitrectomy probes typically include a hollow outer cutting member, a hollow inner cutting member arranged coaxially with and movably disposed within the hollow outer cutting member, and a port extending radially through the outer cutting member near the distal end thereof. Vitreous humor is aspirated into the open port and the inner member is actuated, closing the port. Upon the closing of the port, cutting surfaces on both the inner and outer cutting members cooperate to cut the vitreous, and the cut vitreous is then aspirated away through the inner cutting member. U.S. Pat. Nos. 4,577,629 (Martinez); 5,019,035 (Missirlian, et al.); 4,909,249 (Akkas, et al.); 5,176,628 (Charles, et al.); 5,047,008 (de Juan, et al.); 4,696,298 (Higgins, et al.); and 5,733,297 (Wang) all disclose various types of vitrectomy probes and each of these patents is incorporated in its entirety herein by reference.

Conventional vitrectomy probes include “guillotine style” probes and rotational probes. A guillotine style probe has an inner cutting member that reciprocates along its longitudinal axis. A rotational probe has an inner cutting member that reciprocates around its longitudinal axis. In both types of probes, the inner cutting members are actuated using various methods. For example, the inner cutting member can be moved from the open port position to the closed port position by pneumatic pressure against a piston or diaphragm assembly that overcomes a mechanical spring. Upon removal of the pneumatic pressure, the spring returns the inner cutting member from the closed port position to the open port position. As another example, the inner cutting member can be moved from the open port position to the closed port position using a first source of pneumatic pressure, and then can be moved from the closed port position to the open port position using a second source of pneumatic pressure. As a further example, the inner cutting member can be electromechanically actuated between the open and closed port positions using a conventional rotating electric motor or a solenoid. U.S. Pat. No. 4,577,629 provides an example of a guillotine style, pneumatic piston/mechanical spring actuated probe. U.S. Pat. Nos. 4,909,249 and 5,019,035 disclose guillotine style, pneumatic diaphragm/mechanical spring actuated probes. U.S. Pat. No. 5,176,628 shows a rotational dual pneumatic drive probe.

Each of the above-described conventional vitrectomy probes must have the inner cutting member and outer cutting member aligned to maximize the tissue cutting ability of the probe. Typically, a bend in the inner cutting member creates a preferred cutting edge on the inner cutting member tip. This preferred edge is preferably centered within the port opening of the outer cutting member. To effectuate this alignment, prior art vitrectomy probes use a gate mark on a piston during manufacturing to manually orient the inner cutting member within a fixture that produces the tube bend and the preferred cutting edge. This gate mark is then used during manual assembly of the inner cutting member into the outer cutting member to obtain actual alignment.

Some negative aspects exists with regard to this prior art alignment method. For example, there is a significant manufacturing tolerance associated with the manual alignment of the piston gate mark to the fixture that produces the cutting edge on the inner cutting member. Further, the gate mark is typically large and therefore has an imprecise radial resolution (on the order of, for example, 35°). Further still, once the probe is assembled, the gate mark is inside the assembly and is not visible for verification of proper alignment of the inner cutting member to the outer cutting member. After assembly, the only indication of accurate alignment is probe performance during a cut test, which adds time and expense to the process.

A further disadvantage of prior art vitrectomy probes is illustrated in that typically ophthalmic surgeons do not have simple tools available to measure feature size or fluid depth within the eye during a surgical procedure. Surgeons instead often estimate dimensions using “probe diameters” because it is the only constant reference, inaccurate as it is, within their field of view. Further, some vitrectomy probes, such as those disclosed in U.S. Pat. Nos. 4,909,249 and 5,019,035, have manually adjustable port openings. Manually adjusting the port opening, however, can be time consuming and awkward and is made further so because these probes do not include an objective reference to gauge the port opening size.

Therefore, a need exists for a calibrated surgical probe that can reduce or eliminate the problems of imprecise alignment of the inner and outer cutting members, lack of a length reference during an ophthalmic surgical procedure and lack of an objective reference to judge adjustable port opening size associated with prior art vitrectomy probes.

### BRIEF SUMMARY OF THE INVENTION

The embodiments of the calibrated surgical cutter of the present invention substantially meet these needs and others. One embodiment of this invention is a microsurgical probe tip, comprising: an outer cutting member, comprising a first tube having a wall, a closed end and a port formed in the wall near the closed end; an inner cutting member, comprising a second tube coaxial with and operable to move in a reciprocating motion within the first tube and having a first end operable to be coupled to a driving mechanism and a second end with a cutting edge for cutting tissue; a first alignment mark on the outer cutting member at a first predetermined position adjacent to the port; and a second alignment mark on the inner cutting member at a second predetermined position adjacent to the cutting edge of the inner cutting member, wherein the second alignment mark is visible through the port and operable to be aligned with the first alignment mark such that when the first and second alignment marks are aligned, a preferred relative positioning between the inner and outer cutting members is achieved. The microsurgical probe tip can further comprise one or more radial alignment marks on the inner cutting member, wherein the radial alignment marks are parallel to one another at fixed intervals from one another and positioned so that one or more of the radial alignment marks are visible through the port so as to indicate the relative lateral positioning between the inner cutting member and the outer cutting member.

The relative lateral positioning between the inner cutting member and the outer cutting member indicated by the radial alignment marks can be used to determine an opening amount for the port. The radial alignment marks can be made by a method, or combination of methods, such as laser cutting, inkjet printing, and mechanical scribing. The driving mechanism can be a pneumatic driving mechanism, an electro-mechanical driving mechanism, and/or a magnetic driving mechanism. The cutting edge can comprise a preferred cutting edge and the preferred relative positioning between the inner and outer cutting members can comprises radially centering the preferred cutting edge within the port. The inner cutting member can reciprocate either along the probe tip longitudinal axis or around the probe tip longitudinal axis.

The microsurgical probe tip can further comprising a plurality of gauge marks on an outer surface of the outer cutting member, wherein the gauge marks are parallel to one another at a fixed interval (e.g., 1 millimeter) from one another and positioned so that the gauge marks can be used as a measuring tool in a surgical environment. Further, the first and second alignment marks can be used to align the inner and outer cutting members during assembly of the microsurgical probe tip. The alignment marks, radial alignment marks and gauge marks can be marks comprising a continuous line, a plurality of dots, and/or a row of dashes and they can be of a color contrasting with the color of the inner and outer cutting members.

Other embodiments of the present invention can include a method for aligning the inner and outer cutting members of a surgical probe tip in accordance with the teachings of this invention, a method for measuring features or depths within a surgical site in accordance with the teachings of this invention and a surgical handpiece incorporating the calibrated microsurgical probe of the present invention for use in ophthalmic or other surgery. The embodiments of this invention can be implemented as a handpiece, having a housing for directing and manipulating the calibrated surgical probe in accordance with the teachings of this invention. Further, embodiments of this invention can be incorporated within a surgical machine or system for use in ophthalmic or other surgery, and, in particular, in vitro-retinal surgery. Other uses for a calibrated surgical probe designed in accordance with the teachings of this invention will be known to those familiar with the art.

### BRIEF DESCRIPTION OF THE SEVERAL VIEWS OF THE DRAWINGS

A more complete understanding of the present invention and the advantages thereof may be acquired by referring to the following description, taken in conjunction with the accompanying drawings, in which like reference numbers indicate like features and wherein:

FIGS. 1 and 2 illustrate the distal end of a vitrectomy probe suitable for an embodiment of the present invention, shown in the fully open port position, and closed port position, respectively.

FIG. 3 is a close-up view of the distal end of a vitrectomy probe 10 according to one embodiment of the present invention;

FIG. 4 is a close-up view of a tubular outer cutting member 12 having gauge marks 32 in accordance with one embodiment of this invention;

FIG. 5 shows a close-up view of another embodiment of a vitrectomy probe 10 in accordance with the present invention; and

FIG. 6 shows a simplified block diagram of certain portions of the electronic and pneumatic sub-assemblies of a micro-surgical system 50 which can comprise an embodiment of this invention.

### DETAILED DESCRIPTION OF THE INVENTION

Preferred embodiments of the present invention are illustrated in the FIGUREs, like numerals being used to refer to like and corresponding parts of the various drawings.

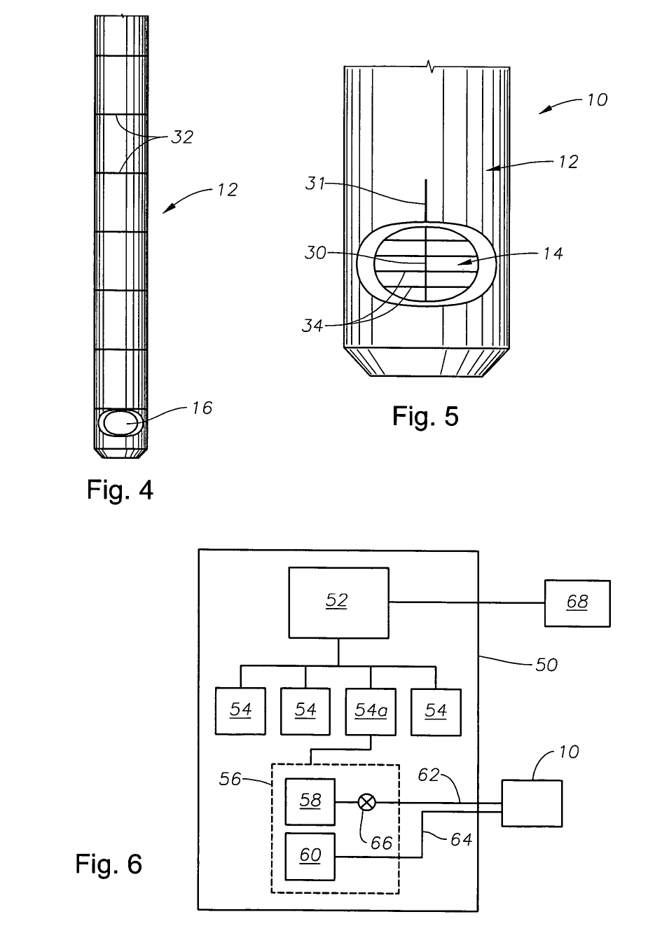

The various embodiments of the present invention provide for a calibrated surgical probe for use in surgical procedures, such as in vitro-retinal/posterior segment surgery. Embodiments of this invention can comprise a vitrectomy probe tip with alignment marks on an internal cutting member and/or an external cutting member, as shown in FIG. 3. Other embodiments of this invention can comprise a vitrectomy probe tip having measurement gauge marks on the outer cutting member, as shown in FIG. 4. Further embodiments can include radial marks on the inner cutting member near the cutting edge to indicate the port opening size, as shown in FIG. 5. Embodiments of the calibrated surgical probe tip of this invention can be configured for use in the general field of ophthalmic surgery. However, it is contemplated and it will be realized by those skilled in the art that the scope of the present invention is not limited to ophthalmology, but may be applied generally to other areas of surgery where precise cutting and/or removal of tissue may be required.

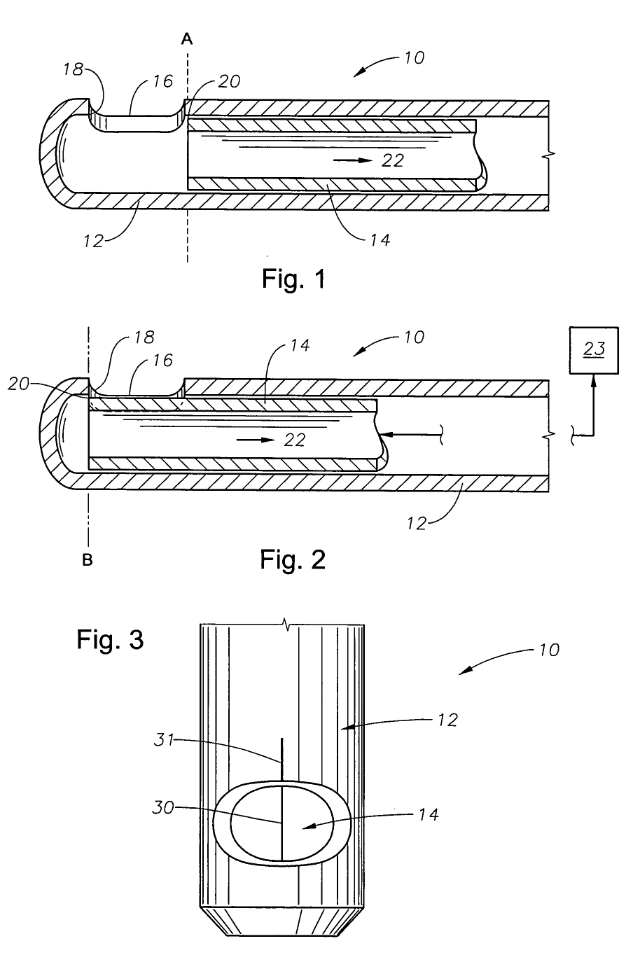

FIGS. 1 and 2 schematically illustrate a distal end of a micro-surgical instrument 10 (micro-surgical probe) tip. Micro-surgical instrument 10 is preferably a guillotine style vitrectomy probe and includes a tubular outer cutting member 12 and a tubular inner cutting member 14 movably disposed within outer cutting member 12. Outer cutting member 12 has a port 16 and a cutting edge 18. Port 16 may typically have a length of about 0.02″ along the longitudinal axis of probe 10. Inner cutting member 14 has a cutting edge 20.

During operation of probe 10, inner cutting member 14 is moved along the longitudinal axis of a probe 10 tip from a position A as shown in FIG. 1 to a Position B as shown in FIG. 2 and then back to Position A in a single cut cycle comprising a reciprocating motion. Position A corresponds to a fully open position of port 16 and Position B corresponds to a fully closed position of port 16. The reciprocating motion is provided by a driving mechanism 23. In a rotational probe 10 tip, inner cutting member 14 would reciprocate around the longitudinal axis of a probe 10 tip. In Position A, vitreous humor or other tissue can be aspirated into port 16 and within inner cutting member 14 by vacuum induced flow represented by arrow 22. In Position B, the vitreous within port 16 and inner cutting member 14 is cut or severed by cutting edges 18 and 20 and is aspirated away by vacuum induced fluid flow 22. Cutting edges 18 and 20 are preferably formed in an interference fit to ensure cutting of the vitreous. In addition, positions A and B are conventionally located somewhat outside the ends of port 16 to account for variations in the actuation of inner cutting member 14 and specific probes 10. Further, in some vitrectomy probes 10, the size of the port 16 fully open position can be varied by varying the inner cutting member 14 stroke.

Although the present invention is described with reference to a pneumatically actuated (pneumatic driving mechanism) vitrectomy probe 10, the principles encompassed in the present invention are equally applicable to micro-surgical instruments having an electro-mechanical or magnetic driving mechanism to move the inner cutting member between the open and closed port positions using, for example, a conventional linear motor or solenoid. For purpose of illustration, and not by way of limitation, the present invention will be described herein with reference to a guillotine style pneumatic/mechanical spring actuated vitrectomy probe 10. Further, the embodiments of the present invention can be incorporated within micro-surgical instruments, such as vitrectomy probe 10, in which the port 16 size and port 16 open duty cycles can be varied, such as with cut-rate. For example, at high cut rates, variations of the duty cycle can facilitate the variation of the “open” size or aperture of port 16. In this way, it is possible to vary the open size of port 16 according to the size of the vitreous or other tissue for cutting and removal. The calibration marks of the embodiments of the present invention serve as an aid to determine and verify the variation in port 16 size opening. The concepts of dynamically varying the duty cycle and/or the open port size with cut rate, and the resulting benefits, are more fully discussed in U.S. Pat. No. 6,773,445, the contents of which are hereby incorporated by reference.

FIG. 3 is a close-up view of the distal end of a vitrectomy probe 10 tip having the calibration/alignment marks of the present invention, on both inner cutting member 14 and outer cutting member 12. As can be seen in FIG. 3, inner alignment mark 30 provides for aligning the cutting edge of the inner cutting member 14 to a predetermined (e.g., most efficient position) within outer cutting member 12, indicated by corresponding outer alignment mark 31 (center of port 16). Alignment marks 30 and 31 can be laser cut (laser marking), inkjet printed, mechanically scribed or made by any other marking technique known to those familiar with the art. A preferred embodiment of the present invention uses laser marking to make alignment marks 30 and 31.

Typically, a bend in the inner cutting member 14 of a vitrectomy probe 10 tip creates a preferred cutting edge along the inner cutting member 14 tube tip. To maximize the tissue cutting ability of the vitrectomy probe 10, this preferred cutting edge should be centered within the port 16 opening of the outer cutting member 12. Alignment mark 30 provides for aligning the preferred cutting edge of inner cutting member 14 to the center of port 16 (alignment mark 31) during the probe 10 assembly. Alignment marks 30 and 31 will enable better cutting edge alignment and provide a means of alignment verification. Aligning inner cutting member 14 to outer cutting member 12 in this manner can improve angular resolution between the inner cutting member 14 edge and the cutting port 16, improve product quality, decrease manufacturing time and reduce the cost of producing vitrectomy probes 10 by improving product yield. Correct alignment can be inspected by advancing the inner cutting member 14 so that its tip is visible through the port 16 and comparing the relative positions of alignment marks 30 and 31. Alignment of the cutting members 12 and 14 during assembly of vitrectomy port 10 can thus be made with greater precision and accuracy.

FIG. 4 is a close-up view of a tubular outer cutting member 12 of a vitrectomy probe 10 tip, having gauge marks 32 spaced, in this example, 1 mm apart. Gauge marks 32 can serve as a simple tool for, for example, ophthalmic surgeons to measure features or fluid depths within an eye during a surgical procedure. Prior art probes do not provide surgeons with such a simple measuring tool and, as a result, some surgeons have had to estimate dimensions using “probe diameters” because it is the only constant reference within their field of view. Gauge marks 32 on a vitrectomy probe 10 in accordance with the present invention will place a standard length reference (e.g., millimeter scale) within the eye which a surgeon can use to measure features in any direction. Gauge marks 32 can also be used by a surgeon to measure depths of therapeutic fluids or gases injected into the eye.

FIG. 5 illustrates another embodiment of a vitrectomy probe 10 tip having alignment marks in accordance with this invention. FIG. 5 shows a close-up view of the distal end of a vitrectomy probe 10 tip, including the outer cutting member 12 and inner cutting member 14. In this embodiment, inner cutting member 14 includes an alignment mark 30, as discussed above, and additional radial alignment marks 34. Radial alignment marks 34 mark the inner cutting member 14 tip to indicate the port 16 opening size in vitrectomy probes 10 having an adjustable port 16 size. This is because one or more radial alignment marks will be visible through the port 16 and the number visible can indicate the relative lateral positioning between the inner cutting member 14 and outer cutting member 12. For example, with a port opening of 0.025″, five radial marks 34, each 0.001″ wide, can be used to clearly indicate 20% increments of port 16 opening size. In this way, surgeons can adjust the port 16 opening to more efficiently and safely remove material to take into account variations in the size, shape, etc., of the vitreous or other tissue targeted for cutting and removal. It is contemplated and within the scope of the present invention that a vitrectomy probe 10 tip can include any combination of alignment marks 30 and 31, gauge marks 32 and radial marks 34 to take advantage of the benefits provided by each. Further, any of alignment marks 30/31, gauge marks 32 and radial marks 34 can be made such that the surface marked remains substantially unchanged to ensure proper mating and motion between the cutting members 12 and 14 so as not to interfere with their designed operation.

FIG. 6 shows a block diagram of certain portions of the electronic and pneumatic sub-assemblies of a micro-surgical system 50 which can incorporate various embodiments of the present invention. For example, system 50 could be the ACCURUS® Surgical System sold by Alcon Laboratories, Inc. of Fort Worth, Tex. or any other conventional ophthalmic micro-surgical system. System 50 preferably includes a host micro-computer 52 that is electronically connected to a plurality of micro-controllers 54. For example, micro-computer 52 can comprise an Intel® Pentium® or other processor and micro-controllers 54 can comprise an Intel® 80/C 196 microprocessor. Of course, other conventional microprocessors having equivalent or superior performance can be utilized for micro-computers 52 and micro-controllers 54 if desired. In FIG. 5, an exemplary micro-controller 54a is shown electronically connected to and controlling an air/fluid module 56 of system 50. Air/fluid module 56 preferably includes a source of pneumatic pressure 58 and a source of vacuum 60, both of which are in fluid communication with a probe 10 via conventional PCV tubing 62 and 64. Air/fluid module 56 also preferably includes appropriate electrical connections between its various components.

Pneumatic pressure source 58 provides pneumatic drive pressure to probe 10. A solenoid valve 66 is disposed within tubing 62 between pneumatic pressure source 58 and probe 10. System 50 also includes a variable controller 68. Variable controller 68 is electronically connected to and controls solenoid valve 66 via microcomputer 52 and micro-controller 54a. Variable controller 68 provides a variable electrical signal that cycles solenoid valve 66 between open and closed positions to provide a cycled pneumatic pressure that drives inner cutting member 14 of probe 10 from its open port position to its closed port position at a variety of cut rates. Variable controller 68 can be a conventional footswitch or foot pedal that is operable by a surgeon. For example, variable controller 68 may be the foot pedal sold as part of the ACCURUS® Surgical System mentioned above. Alternatively, variable controller 68 could also be a conventional handheld switch or “cut screen” control if desired.

From the above it may be appreciated that the embodiments of the present invention provide an improved method of aligning a preferred cutting edge of a micro-surgical cutter with a port opening of the micro-surgical cutter, a simple means to measure features within the eye during a surgical procedure by providing a standard length reference, and a means to indicate port opening size in vitrectomy probes having an adjustable port, that provide significant benefits to both the surgeon and the patient. The embodiments of the calibrated surgical cutter tip of the present invention thus provide various advantages over prior art surgical cutters. For example, alignment marks 30 and 31 can provide a means for fixing the relationship between the alignment mark 30 and the cutting edge of inner cutting member 14 by allowing the use of the same manufacturing fixture for making alignment mark 32 and for forming of the cutting edge 20 of inner cutting member 14. Further, alignment mark 30 provides superior radial resolution over the prior art. For example, alignment mark 30 can be a laser mark having a width of 0.001″. For a 25 gauge vitrectomy probe tip, the inner cutting member diameter is typically equal to 0.015″, the circumference is equal to the product of π and the diameter (i.e., 0.047″) and radial resolution is thus equal to 0.001″ divided by 0.047 which is equal to 2.1%. Thus, 0.021 multiplied by 360° results in a radial resolution equal to 7.6°. Similarly, for a 20 gauge cutter having a diameter of 0.025″, radial resolution will be to 4.7°. A further advantage provided by alignment marks 30 and 31 is that the alignment of the inner cutting member and the outer cutting member tubes can be automated using computer vision to further improve alignment consistency in a manner that will be known to those familiar with the art. Once a vitrectomy probe 10 is assembled, alignment marks 30 and 31 can be seen and used for both post insertion adjustment, if required, and for confirmation of correct final position.

Gauge marks 32 provide similar advantages for both the surgeon and patient in that they provide a standard length reference for the surgeon within the eye. Gauge functionally is a bonus feature provided by the embodiments of this invention because a vitrectomy probe is already present within the eye during the surgical procedure. As such, gauge marks 32 can provide surgeons with a “dip stick” to measure the depth of therapeutic fluids or gases injected into the eye. As noted above, the various embodiments of the present invention are contemplated for use with any gauge surgical probe, and in particular with 20 gauge and 25 gauge vitrectomy cutters.

One embodiment of a vitrectomy probe 10 tip can use, as a base material for the inner and outer cutting members 12 and 14, 304 stainless steel that is double drawn (for hardness) having an outer diameter equal to 0.0146″ and an inner diameter equal to 0.01″, resulting in a nominal wall of 0.0023″. The base material can be coated with titanium nitride for hardness and lubrication, having a typical thickness of 0.0001″. In this case, the appearance of the cutting assemblies is gold colored. In accordance with the teachings of the present invention, calibration marks 30, 31, 32 and 34 provide for proper alignment of inner cutting member 14 within a larger outer cutting member 12 having a port 16. Alignment mark width can be very thin, on the order of about 0.001″ or less, to provide good radial resolution in the vitrectomy probe assembly. Alignment mark 30, for example, can be oriented to start 0.002″ from the cutting edge of the inner cutting member 14, so as not to affect the cutting edge, and can be about 0.01″ long. Alignment marks 30, 31, 32, and 34 can comprise a continuous line, a row of dots, a row of dashes or any other such configuration as is known to those familiar with the art. Similarly, the marks can be white, black, silver or any other such color that can provide sufficient contrast to the background material on which the marks are made, such that it is possible to see the marks using magnified human vision, or in the case of assembly, machine vision. Visibility of the marks should be such that they can be easily seen on bare or coated material, such as the 304 stainless steel or titanium nitride coating discussed above. Adjustments can be made in the method of making marks 30, 31, 32 and 34 such that proper contrast is provided regardless of the material.

The present invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims.

What is claimed is:

1. A microsurgical probe tip, comprising:
an outer cutting member, comprising a first tube having a wall, a closed end and a port formed in the wall near the closed end;
an inner cutting member, comprising a second tube coaxial with and operable to move in a reciprocating motion within the first tube and having a first end operable to be coupled to a driving mechanism and a second end with a cutting edge for cutting tissue;
a first alignment mark on the outer cutting member at a first predetermined position adjacent to the port; and
a second alignment mark on the inner cutting member at a second predetermined position adjacent to the cutting edge of the inner cutting member, wherein the second alignment mark is visible through the port and operable to be aligned with the first alignment mark such that when the first and second alignment marks are aligned, a preferred relative positioning between the inner and outer cutting members is achieved.

2. The microsurgical probe tip of claim 1, further comprising one or more radial alignment marks on the inner cutting member, wherein the radial alignment marks are parallel to one another at fixed intervals from one another and positioned so that one or more of the radial alignment marks are visible through the port so as to indicate the relative lateral positioning between the inner cutting member and the outer cutting member.

3. The microsurgical probe tip of claim 2, wherein the relative lateral positioning between the inner cutting member and the outer cutting member indicated by the radial alignment marks is used to determine an opening amount for the port.

4. The microsurgical probe tip of claim 2, wherein the radial alignment marks are made by a method selected from the group consisting of laser cutting, inkjet printing, and mechanical scribing.

5. The microsurgical probe tip of claim 1, wherein the microsurgical probe tip is an ophthalmic microsurgical probe tip.

6. The microsurgical probe tip of claim 1, wherein the microsurgical probe tip is a vitrectomy probe tip.

7. The microsurgical probe tip of claim 1, wherein the first and second alignment marks are made by a method selected from the group consisting of laser cutting, inkjet printing, and mechanical scribing.

8. The microsurgical probe tip of claim 1, wherein the driving mechanism is selected from one of the group consisting of a pneumatic driving mechanism, an electro-mechanical driving mechanism, and a magnetic driving mechanism.

9. The microsurgical probe tip of claim 1, wherein the cutting edge comprises a preferred cutting edge and wherein the preferred relative positioning between the inner and outer cutting members comprises radially centering the preferred cutting edge within the port.

10. The microsurgical probe tip of claim 9, wherein the second predetermined position indicates the preferred cutting edge.

11. The microsurgical probe tip of claim 1, wherein the first predetermined position indicates the radial center of the port.

12. The microsurgical probe tip of claim 1, wherein the inner cutting member's reciprocating motion is reciprocating motion along the probe tip longitudinal axis.

13. The microsurgical probe tip of claim 1, wherein the inner cutting member's reciprocating motion is reciprocating motion around the probe tip longitudinal axis.

14. The microsurgical probe tip of claim 1, further comprising a plurality of gauge marks on an outer surface of the outer cutting member, wherein the gauge marks are parallel to one another at a fixed interval from one another and positioned so that the gauge marks can be used as a measuring tool in a surgical environment.

15. The microsurgical probe tip of claim 14, wherein the fixed interval is 1 millimeter.

16. The microsurgical probe tip of claim 1, wherein the first and second alignment marks have a width of about 0.001 inch or less and about 0.01 inches in length.

17. The microsurgical probe tip of claim 1, wherein the first and second alignment marks are used to align the inner and outer cutting members during assembly of the microsurgical probe tip.

18. The microsurgical probe tip of claim 1, wherein the probe tip is a 20 to 25 gauge tip.

19. The microsurgical probe tip of claim 1, wherein the first and second alignment marks comprise a mark selected from the group consisting of a continuous line, a plurality of dots, and a row of dashes.

20. The microsurgical probe tip of claim 1, wherein the first and second alignment marks are a color contrasting with the color of the inner and outer cutting members.

21. A microsurgical probe tip, comprising:
an outer cutting member, comprising a first tube having a wall, a closed end and a port formed in the wall near the closed end;
an inner cutting member, comprising a second tube coaxial with and operable to move in a reciprocating motion within the first tube and having a first end operable to be coupled to a driving mechanism and a second end with a cutting edge for cutting tissue; and
one or more radial alignment marks on the inner cutting member, wherein the radial alignment marks are parallel to one another at fixed intervals from one another and positioned so that one or more of the radial alignment marks are visible through the port so as to indicate the relative lateral positioning between the inner cutting member and the outer cutting member.

22. The microsurgical probe tip of claim 21, wherein the relative lateral positioning between the inner cutting member and the outer cutting member indicated by the radial alignment marks is used to determine an opening amount for the port.

23. The microsurgical probe tip of claim 21, wherein the radial alignment marks are made by a method selected from the group consisting of laser cutting, inkjet printing, and mechanical scribing.

24. The microsurgical probe tip of claim 21, wherein the microsurgical probe tip is an ophthalmic microsurgical probe tip.

25. The microsurgical probe tip of claim 21, wherein the microsurgical probe tip is a vitrectomy probe tip.

26. The microsurgical probe tip of claim 21, wherein the radial alignment marks have a width of about 0.001 inch or less and a length of about 0.01 inch.

27. The microsurgical probe tip of claim 21, wherein the probe tip is a 20 to 25 gauge tip.

28. The microsurgical probe tip of claim 21, wherein each of the radial alignment marks comprise a mark selected from the group consisting of a continuous line, a plurality of dots, and a row of dashes.

29. The microsurgical probe tip of claim 21, wherein the radial alignment marks are a color contrasting with the color of the inner and outer cutting members.

30. A microsurgical probe tip, comprising:
an outer cutting member, comprising a first tube having a wall, a closed end and a port formed in the wall near the closed end;
an inner cutting member, comprising a second tube coaxial with and operable to move in a reciprocating motion within the first tube and having a first end operable to be coupled to a driving mechanism and a second end with a cutting edge for cutting tissue; and
a plurality of gauge marks on an outer surface of the outer cutting member, wherein the gauge marks are parallel to one another at a fixed interval from one another and positioned so that the gauge marks can be used as a measuring tool in a surgical environment.

31. The microsurgical probe tip of claim 30, wherein the fixed interval is 1 millimeter.

32. The microsurgical probe tip of claim 30, wherein the gauge marks are made by a method selected from the group consisting of laser cutting, inkjet printing, and mechanical scribing.

33. The microsurgical probe tip of claim 30, wherein the microsurgical probe tip is an ophthalmic microsurgical probe tip.

34. The microsurgical probe tip of claim 30, wherein the microsurgical probe tip is a vitrectomy probe tip.

35. The microsurgical probe tip of claim 30, wherein the gauge marks have a width of about 0.001 inch or less.

36. The microsurgical probe tip of claim 30, wherein the probe tip is a 20 to 25 gauge tip.

37. The microsurgical probe tip of claim 30, wherein each of the gauge marks comprise a mark selected from the group consisting of a continuous line, a plurality of dots, and a row of dashes.

38. The microsurgical probe tip of claim 30, wherein the gauge marks are a color contrasting with the color of the inner and outer cutting members.
